(19) United States
(12) Patent Application Publication
(10) Pub. No.: US 2025/0295705 A1
(43) Pub. Date: Sep. 25, 2025

(54) FOOD AND MEDICINE COMPONENT COMPOSITION, FOOD AND MEDICINE COMPOUND MEDICAMENT, AND PREPARATION METHOD OF FOOD AND MEDICINE COMPOUND MEDICAMENT AND USE THEREOF

(71) Applicant: NATIONAL RESEARCH INSTITUTE OF CHINESE MEDICINE, MINISTRY OF HEALTH AND WELFARE, Taipei (TW)

(72) Inventors: YI-CHANG SU, Taipei (TW); WEN-HUI CHIOU, Taipei (TW); YUH-CHIANG SHEN, Taipei (TW); KUO-TONG LIOU, Taipei (TW); CHUNG-KUANG LU, Taipei (TW); YUNG-CHI HSIAO, Taipei (TW); WEN-CHI WEI, Taipei (TW); KENG-CHANG TSAI, Taipei (TW); CHIA-CHING LIAO, Taipei (TW); YI-CHIA HUANG, Taipei (TW); CHUNG-HSING CHOU, Taipei (TW)

(73) Assignee: NATIONAL RESEARCH INSTITUTE OF CHINESE MEDICINE, MINISTRY OF HEALTH AND WELFARE, Taipei (TW)

(21) Appl. No.: 19/086,154

(22) Filed: Mar. 21, 2025

Related U.S. Application Data
(60) Provisional application No. 63/567,957, filed on Mar. 21, 2024.

(30) Foreign Application Priority Data
Mar. 3, 2025 (TW) 114107712

(51) Int. Cl. A61K 35/60; A61K 36/236; A61K 36/48; A61K 36/65; A61P 25/28

(52) U.S. Cl. CPC A61K 35/60; A61K 36/236; A61K 36/48; A61K 36/65; A61P 25/28; A61K 2236/331; A61K 2236/37

(57) ABSTRACT

A food and medicine component composition, a food and medicine compound medicament, and a preparation method of a food and medicine compound medicament and use thereof are provided. The food and medicine component composition includes 0.5 to 1.5 parts by weight of Red Peony Root; 0.5 to 1.5 parts by weight of Chuanxiong Rhizome; 10 to 15 parts by weight of Hedysarum Root; and 50 to 55 parts by weight of whole raw fish. The food and medicine compound medicament is prepared by the following preparation method: placing the above food and medicine component composition in water, and heating for decoction to obtain the food and medicine compound medicament. The food and medicine compound medicament prepared from the above food and medicine component composition can be used for preparing a drug for treating acute central nervous system injury or a sequela of acute central nervous system injury.

### CROSS-REFERENCE TO RELATED APPLICATION

This non-provisional application claims priority under 35 U.S.C. § 119 (e) on U.S. provisional Patent Application No(s). 63/567,957 filed on Mar. 21, 2024, the entire contents of which are hereby incorporated by reference.

This non-provisional application claims priority under 35 U.S.C. § 119 (a) on Patent Application No(s). 114107712 filed in Taiwan, R.O.C. on Mar. 3, 2025, the entire contents of which are hereby incorporated by reference.

### BACKGROUND OF THE INVENTION

### 1. Field of the Invention

The present invention relates to a food and medicine component composition, a food and medicine compound medicament, and a preparation method of a food and medicine compound medicament and use thereof, and in particular to a food and medicine component composition and a food and medicine compound medicament that can be used for treating acute central nervous system injury or a sequela thereof.

### 2. Description of the Related Art

Acute central nervous system injury is a serious disease caused by sudden damage to the central nervous system of the human body. Common types of acute central nervous system injury include acute stroke, traumatic brain injury, spinal cord injury, etc., among which acute central nervous system injury induced by acute stroke is the most common. Acute stroke is an acute condition that occurs due to obstruction or rupture of blood vessels that supply oxygen and nutrients to the brain, resulting in the inability of brain tissues to receive normal blood flow. When patients suffer from acute stroke, even if the patients receive immediate treatment, they will still have sequelae such as limb movement disorders (e.g., limb paralysis, dysphagia) or cognitive impairment due to impaired brain function.

At present, the main treatment methods for the sequelae caused by acute central nervous system injury such as acute stroke include physical therapy and occupational therapy. That is, by guiding patients to carry out continuous rehabilitation training, the remodeling and recovery of their cranial nerves can be strengthened, so that the limb movement disorders or cognitive impairment caused by acute central nervous system injury in the patients can be gradually improved.

### BRIEF SUMMARY OF THE INVENTION

As described above, the existing main treatment methods for the sequelae of acute central nervous system injury include physical therapy and occupational therapy. The above physical therapy and occupational therapy require a relatively long time and human intervention to achieve better treatment and recovery effects. Therefore, it is desirable to propose a drug capable of more effectively improving the sequelae of acute central nervous system injury.

An objective of the present invention is to solve the above problems by providing a food and medicine component composition, including: 0.5 to 1.5 parts by weight of Red Peony Root; 0.5 to 1.5 parts by weight of Chuanxiong Rhizome; 10 to 15 parts by weight of Hedysarum Root; and 50 to 55 parts by weight of whole raw fish.

In the food and medicine component composition as described above, the whole raw fish is selected from the group consisting of Monopterus and Anguilla.

In the food and medicine component composition as described above, a species of the whole raw fish is Monopterus albus.

In the food and medicine component composition as described above, parts by weight of the whole raw fish are 50 to 55 parts by weight.

In order to achieve the above objective and other objectives, the present invention provides a method for preparing a food and medicine compound medicament, including: (a) providing 0.5 to 1.5 parts by weight of Red Peony Root, 0.5 to 1.5 parts by weight of Chuanxiong Rhizome, 10 to 15 parts by weight of Hedysarum Root, 50 to 55 parts by weight of whole raw fish and 200 to 400 parts by weight of water; (b) soaking the Red Peony Root, the Chuanxiong Rhizome, the Hedysarum Root and the whole raw fish in the water, and heating the water to boiling to perform decocting treatment on the Red Peony Root, the Chuanxiong Rhizome, the Hedysarum Root and the whole raw fish; and (c) boiling the water provided in step (a) to approximately 75% by volume of an initial volume of the water to obtain the food and medicine compound medicament.

In the preparation method as described above, the whole raw fish is selected from the group consisting of Monopterus and Anguilla.

In the preparation method as described above, a species of the whole raw fish is Monopterus albus.

In order to achieve the above objective and other objectives, the present invention provides a food and medicine compound medicament prepared by the preparation method as described above.

In order to achieve the above objective and other objectives, the present invention provides a method for treating acute central nervous system injury or a sequela of acute central nervous system injury, comprising administrating a drug prepared from the food and medicine component composition which includes the food and medicine component composition as described above to a subject in need thereof.

In order to achieve the above objective and other objectives, the present invention provides a method for treating acute central nervous system injury or a sequela of acute central nervous system injury, comprising administrating a food and medicine compound medicament which includes the food and medicine compound medicament as described above to a subject in need thereof.

Through the food and medicine component composition, the food and medicine compound medicament, and the preparation method of a food and medicine compound medicament and the use thereof, a food and medicine component composition and a food and medicine compound medicament capable of more effectively improving a sequela of acute central nervous system injury can be provided.

### BRIEF DESCRIPTION OF THE DRAWINGS

FIG. 1 shows an HPLC spectrum of food and medicine compound medicament NRICM301 in an example of the present invention.

FIG. 2 shows a comparison diagram of survival rates of mice in different groups 1 day after induction of acute stroke.

FIG. 3 shows degrees of walking ability of mice in different groups 1 day after induction of acute stroke.

FIG. 4 shows degrees of nerve injury of mice in different groups 1 day after induction of acute stroke.

FIG. 5 shows degrees of daily living ability of mice in different groups 1 day after induction of acute stroke.

FIG. 6 shows degrees of walking ability of mice in different groups 7 days after induction of acute stroke.

FIG. 7 shows degrees of daily living ability of mice in different groups 7 days after induction of acute stroke.

FIG. 8 shows degrees of learning and memory ability of mice in different groups 7 days after induction of acute stroke.

FIG. 9 shows fluorescent staining images of lateral ventricle sections of treated mice after administering food and medicine compound medicament NRICM301 in an example of the present invention to mice after acute stroke.

FIG. 10 shows fluorescent staining images of lateral ventricle sections of treated mice after administering food and medicine compound medicament NRICM301 in an example of the present invention to mice after acute stroke.

FIG. 11 shows fluorescent staining images of lateral ventricle sections of treated mice after administering food and medicine compound medicament NRICM301 in an example of the present invention to mice after acute stroke.

### DETAILED DESCRIPTION OF THE INVENTION

In order to fully understand the objectives, features and effects of the present invention, the present invention will be described in detail with reference to the following specific example in conjunction with the accompanying drawings. The description is as follows.

Food and medicine component composition of this example and food and medicine compound medicament prepared therefrom:
this example provides a food and medicine component composition that can be prepared into a food and medicine compound medicament for improving the sequelae of acute central nervous system injury (such as acute stroke). The components of the food and medicine component composition and the method for preparing the food and medicine component composition into the food and medicine compound medicament are described in detail below.

First, medicinal materials and fish as shown in Table 1 below were prepared as prescription components of the food and medicine component composition used in this example.

TABLE 1
Prescription components of the food
and medicine component composition

| Prescription component | Medicinal part | Weight | |
|---|---|---|---|
| Hedysarum Root | Dried root | 45.00 | g |
| Red Peony Root | Dried root | 3.75 | g |
| Chuanxiong Rhizome | Dried rhizome | 3.75 | g |
| Monopterus albus | | 200 | g |

The medicinal materials such as Hedysarum Root, Red Peony Root and Chuanxiong Rhizome in Table 1 above were first soaked in 1100 ml of filtered water for 30 minutes. Then the cleaned Monopterus albus (whole raw fish without removal of internal organs and uncooked) was added to the aforementioned medicinal materials and soaked together in the water. The mixture was decocted in an electric cooker with a water-bath method for 120 minutes (the decoction time could be adjusted according to heating conditions, as long as the water could be boiled to 75% by volume of the original volume of the water) to prepare the food and medicine compound medicament. After the decoction was completed, the decocted medicinal liquid was filtered with No. 1 filter paper, and finally, approximately 810 ml of clear filtrate was obtained. The aforementioned clear filtrate was the medicinal liquid of the food and medicine compound medicament in this example. After the aforementioned clear filtrate was lyophilized, approximately 38.32 g of the food and medicine compound medicament in the form of dried powder was obtained. The extraction rate of the aforementioned food and medicine component composition was approximately 15.17%. The food and medicine compound medicament in this example was hereinafter referred to as NRICM301.

### HPLC Spectrum Analysis of NRICM301

First, 50 mg of the lyophilized powder of NRICM301 was taken and added to 1.0 ml of deionized water for redissolution. Then, the aforementioned NRICM301 dissolved in the deionized water was centrifuged at a rotational speed of 13000 rpm for 10 minutes. After the centrifugation was completed, the supernatant was taken as the analysis sample. The aforementioned supernatant was filtered through a 0.22 μm filter screen (made of PVDF material), and then the spectrum of the NRICM301 sample was analyzed with a high-performance liquid chromatograph (HPLC-DAD). The analysis process of the HPLC fingerprint spectrum was performed with reference to the operation manual of the instrument. The instrument model and analysis conditions of the aforementioned high-performance liquid chromatograph are as described in Table 2 below.

TABLE 2
Instrument and analysis conditions of the high-
performance liquid chromatograph (HPLC)

| HPLC model | SHIMADZU LC-2050C | | | | | | | | | |
|---|---|---|---|---|---|---|---|---|---|---|
| Column | ODS COSMOSIL 5C₁₈-AR-II (4.6 mm × 250 mm) | | | | | | | | | |
| model | | | | | | | | | | |
| Solvent | A: acetonitrile (containing 0.1 v/v % phosphoric acid) | | | | | | | | | |
| system | B: deionized water (containing 0.1 v/v % phosphoric acid) | | | | | | | | | |
| Gradient | 0.01 | min | 5.00 | min | 15.00 | min | 40.00 | min | 50.00 | min |
| program | 0% | A | 0% | A | 12% | A | 40% | A | 80% | A |

| | | | | | |
|---|---|---|---|---|---|
| Sample | 50 | mg/ml | Injection volume | 3 | μL |

concentration

| | | | | | |
|---|---|---|---|---|---|
| Flow rate | 1.0 | ml/minute | Column temperature | 40° | C. |

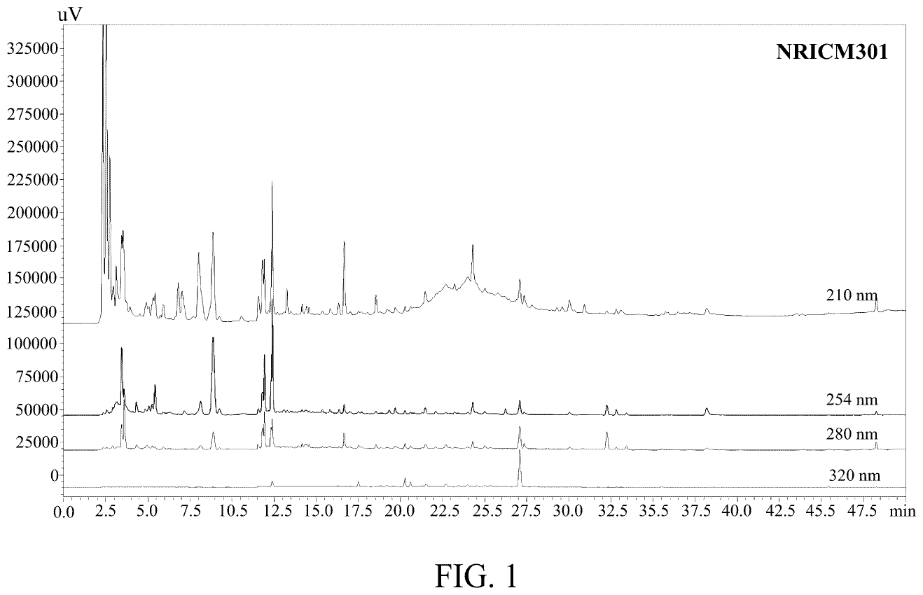

The HPLC spectrum of NRICM301 is shown in FIG. 1. After comparing the active ingredients in the HPLC spectrum, it was confirmed that NRICM301 contained at least three active ingredients, i.e. ferulic acid, paeoniflorin and medicarpin.

In the above formulation of the food and medicine component composition for preparing NRICM301, the Red Peony Root and the Chuanxiong Rhizome were each 1 part by weight, the Hedysarum Root was 12 parts by weight, and the fish was approximately 53 parts by weight. However, in other tests, it was found that when the parts by weight of the Red Peony Root were within the range of 0.5 to 1.5 parts by weight (preferably 0.9 to 1.1 parts by weight), the parts by weight of the Chuanxiong Rhizome were within the range of 0.5 to 1.5 parts by weight (preferably 0.9 to 1.1 parts by weight), the parts by weight of the Hedysarum Root were within the range of 10 to 15 parts by weight (preferably 11 to 13 parts by weight), and the parts by weight of the whole raw fish were within the range of 50 to 55 parts by weight (preferably between 52 and 54 parts by weight), a food and medicine compound medicament with the same active ingredients as the aforementioned NRICM301 could be prepared. Moreover, in this example, Monopterus albus was used as the whole raw fish food material. However, other fish species can also be used as the whole raw fish food material for preparing NRICM301 because they have similar components to Monopterus albus and are rich in various amino acids, which are not limited to this example. For example, the aforementioned whole raw fish food material was preferably selected from the fish species of Monopterus and Anguilla. Furthermore, the amount of the water used for decocting the above medicinal materials and fish in this example was approximately equivalent to 293 parts by weight. In other tests, it was found that when the parts by weight of the water were within the range of 200 to 400 parts by weight, the water could also be used to prepare a food and medicine compound medicament with the same active ingredients.

### Behavioral Observation Test on the Therapeutic Effect of NRICM301 on Acute Stroke

In this test, a behavioral observation test was conducted on the therapeutic effect and sequela recovery effect of NRICM301 in this example and other similar formulations on mice with acute stroke. The test process was carried out as follows.

First, according to the different permutations and combinations of food and medicine components in Table 3 and Table 4 below, the following formulations were provided: BHD-A-01, BHD-MA-01, BHD-O-01, BHD-A-02, BHD-MA-02, BHD-MA-03 (this was the formulation of NRICM301 in this example). In addition, in this test, the ancient prescription BHD with similar components to NRICM301 was also prepared for testing. The medicinal material formulation and weight ratio of BHD were comparable as those of NRICM301 in the present invention. The main difference was that BHD used the traditional Chinese medicine Earthworm (originally the whole body of Pheretima aspergillum, an animal of the Megascolecidae family) instead of fish.

TABLE 3
Weights of medicinal materials in formulations

| Formulation code | Hedysarum Root | | Chuanxiong Rhizome | | Red Peony Root | | Fish |
|---|---|---|---|---|---|---|---|
| 01 | 37.5 g | (one liang) | 3.75 g | (1 qian) | 18.75 g | (5 qian) | 500 g |
| 02 | 37.5 g | (one liang) | 11.25 g | (3 qian) | 18.75 g | (5 qian) | 100 g |
| 03 | 45 g | (12 qian) | 3.75 g | (1 qian) | 3.75 g | (1 qian) | 200 g |

TABLE 4
Species of fish in formulations

| Formulation code | Species |
|---|---|
| BHD-A | Eel |
| BHD-MA | Monopterus albus |
| BHD-O | No fish added |
| BHD-D | Earthworm |

After the medicinal materials and food materials required for each of the formulations BHD-A-01, BHD-MA-01, BHD-O-01, BHD-A-02, BHD-MA-02, BHD-MA-03 and BHD were prepared, the medicament powder of each was then made separately using the method for preparing the food and medicine compound medicament of the above NRICM301.

Then, 10 healthy mice (represented by the code “sham”) and 130 mice subjected to stroke induction treatment were prepared, with 10 mice used in each experimental group. The following were the group codes for this test: sham (the control group, in which no treatment was performed on the mice); CIR (the mice undergoing stroke induction treatment, to which no drug treatment was administered); CIR+BHD (2 g/kg) (2 g/kg of BHD was administered to the stroke mice); BHD-MA-03 (2 g/kg) (2 g/kg of BHD-MA-03 was administered to the stroke mice); BHD-MA-03 (5 g/kg) (5 g/kg of BHD-MA-03 was administered to the stroke mice); BHD-A-01 (1 g/kg) (1 g/kg of BHD-A-01 was administered to the stroke mice); BHD-A-01 (5 g/kg) (5 g/kg of BHD-A-01 was administered to the stroke mice); BHD-A-02 (1 g/kg) (1 g/kg of BHD-A-02 was administered to the stroke mice); BHD-A-02 (2 g/kg) (2 g/kg of BHD-A-02 was administered to the stroke mice); BHD-MA-02 (1 g/kg) (1 g/kg of BHD-MA-02 was administered to the stroke mice); BHD-O-01 (1 g/kg) (1 g/kg of BHD-O-01 was administered to the stroke mice); BHD-MA-01 (5 g/kg) (5 g/kg of BHD-MA-01 was administered to the stroke mice); and BHD-MA-02 (5 g/kg) (5 g/kg of BHD-MA-02 was administered to the stroke mice).

After the mice in each of the above experimental groups were subjected to the stroke induction treatment, then, on the day after the stroke induction treatment, the formulated medicaments in the designed dosages for each group were gavaged to the mice in each group. The drug administration was continued and the condition was observed for 7 days, with the drug administered once a day, and the final survival rate, the degree of walking ability, the degree of nerve injury, the degree of daily living ability and the degree of learning and memory ability of the mice in each group during the 7 days were recorded and tested. The mice in each group were individually housed in cages.

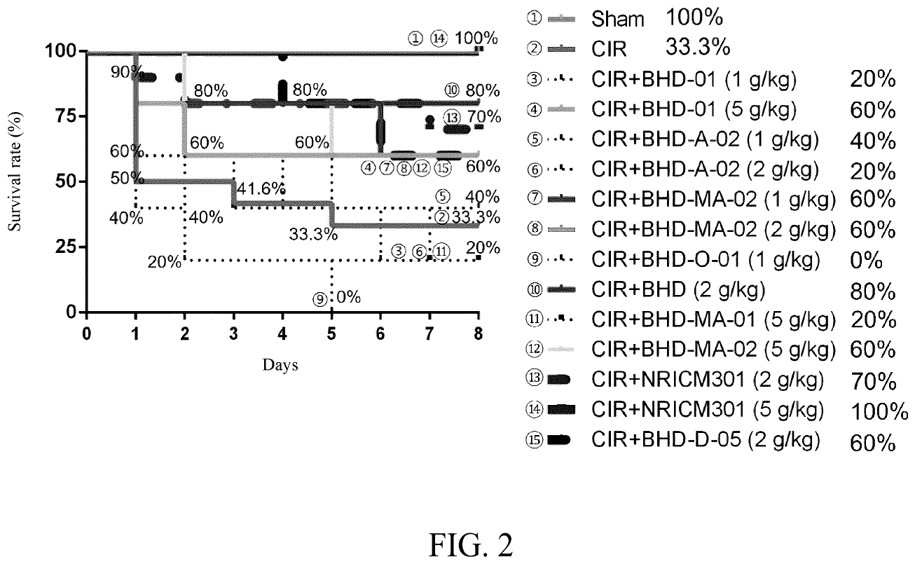

The final survival rate results of the mice in each group after 7 days are shown in FIG. 2. The mice in the three groups of CIR+BHD (2 g/kg), BHD-MA-03 (2 g/kg) and BHD-MA-03 (5 g/kg) were found to have a better survival rate of more than 75%. In the following tests regarding the degree of walking ability, the degree of nerve injury, the degree of daily living ability and the degree of learning and memory ability, the performances of the mice in the 3 groups of CIR+BHD (2 g/kg), BHD-MA-03 (2 g/kg) and BHD-MA-03 (5 g/kg) were further tested and their data were recorded. At the same time, the performances of these mice were compared with those of the mice in the sham group and the CIR group.

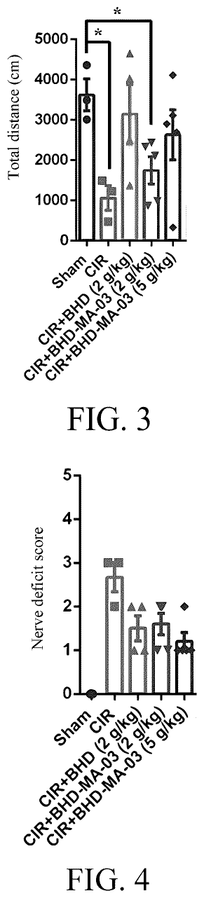

The mice in each group underwent a walking ability test 1 day after being induced with acute stroke (the test was performed on the surviving mice in each group). The test method was described as follows. First, the mice in each group were made to move on the same standard line, and the moving distance of the mice in each group within 5 minutes was recorded. The test results are shown in FIG. 3. The average moving distance of the mice in the sham group could reach approximately 3500 cm, the average moving distance of the mice in the CIR group could reach close to 1000 cm, the average moving distance of the mice in the CIR+BHD (2 g/kg) group could reach 3000 cm, the average moving distance of the mice in the BHD-MA-03 (5 g/kg) group could reach 2800 cm, and the average moving distance of the mice in the BHD-MA-03 (2 g/kg) group could reach 1800 cm. According to the above test results, it could be known that the walking abilities of the mice in the 3 groups of CIR+BHD (2 g/kg) (the ancient prescription), BHD-MA-03 (2 g/kg) (NRICM301) and BHD-MA-03 (5 g/kg) (NRICM301) were significantly restored compared with those of the untreated acute stroke mice. The mice in the CIR+BHD (2 g/kg) group were treated with the ancient prescription, but the medicinal material pheretima used in the ancient prescription is a medicinal material that modern people do not like to use. However, the food and medicine compound medicament NRICM301 provided in this example was changed to use a dietary therapy formulation acceptable to the general public, and at the same time, it still had a therapeutic effect comparable to that of the ancient prescription.

The mice in each group underwent an observation of the degree of nerve injury 1 day after being induced with acute stroke (the test was performed on the surviving mice in each group). The observation method was described as follows. First, the abnormal behavior manifestations of the mice in each group were observed, and the degree of nerve injury of the mice was scored according to their abnormal behavior manifestations. 0 points: no abnormal manifestations; 1 point: unilateral mild lameness; 2 points: tail-chasing rotation; 3 points: epilepsy; and 4 points: paralysis and immobility. The test results are shown in FIG. 4. The degree of nerve injury of the mice in the sham group was 0 points, i.e., no nerve injury at all; the mice in the CIR group which were induced with acute stroke and untreated had a score close to 3 points; the average score of the mice in the CIR+BHD-MA-03 (5 g/kg) (NRICM301) group was about 1 point; the average score of the mice in the CIR+BHD (2 g/kg) (ancient prescription) group was about 1.6 points; and the average score of the mice in the CIR+BHD-MA-03 (2 g/kg) (NRICM301) group was about 1.8 points. According to the above test results, it could be known that NRICM301 had a better therapeutic effect on the nerve injury of acute stroke mice.

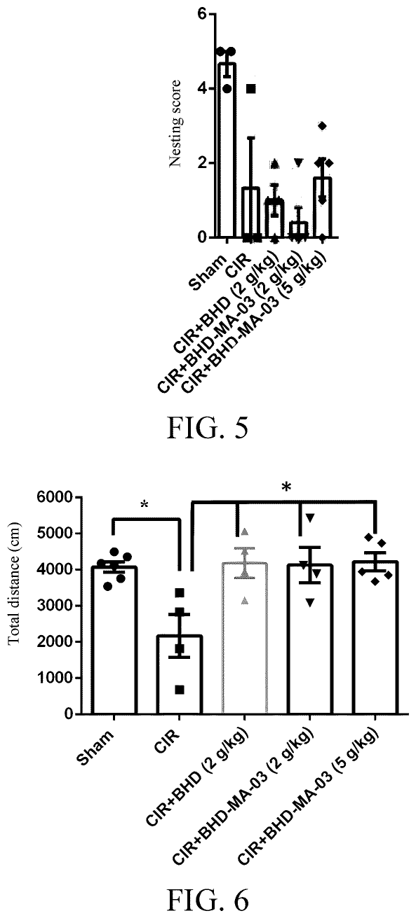

The mice in each group underwent a daily living ability test 1 day after being induced with acute stroke (the test was performed on the surviving mice in each group). The test method was described as follows. First, 2 cotton pads of a fixed weight were given to the mice in each group. 24 hours after the cotton pads were put in, the situation of the mice in each group tearing the cotton pads to build a nest in the cage (nest building is an instinctive behavior of mice) was observed. The most complete nest structure among all the mice was scored as 5 points (full score), and the nest structures of other mice were scored by comparison with the aforementioned most complete nest structure, and the scores were estimated from 1 to 5 points. The test results are shown in FIG. 5. The average score of the mice in the sham group could reach 4.5 points, the average score of the mice in the BHD-MA-03 (5 g/kg) (NRICM301) group could reach 2 points, the average score of the mice in the CIR+BHD (2 g/kg) (ancient prescription) group could reach 1 point, and the average score of the mice in the CIR group was less than 2 points. According to the above test results, it could be known that NRICM301 could still show a significant effect of improving the daily living ability of mice.

The mice in each group underwent a walking ability test 7 days after being induced with acute stroke (the test was performed on the surviving mice in each group). The test method was the same as that of the aforementioned walking ability test. The test results are shown in FIG. 6. The average moving distance of the mice in the sham group could reach approximately 4000 cm. The average moving distances of the mice in the CIR+BHD (2 g/kg) group, the BHD-MA-03 (5 g/kg) group and the BHD-MA-03 (2 g/kg) group were all comparable to that of the mice in the sham group. The average moving distance of the mice in the CIR group was about 2000 cm, which was significantly behind that of the other groups. According to the above test results, it could be known that both NRICM301 and the ancient prescription had a significant effect of improving the walking ability of mice after acute stroke.

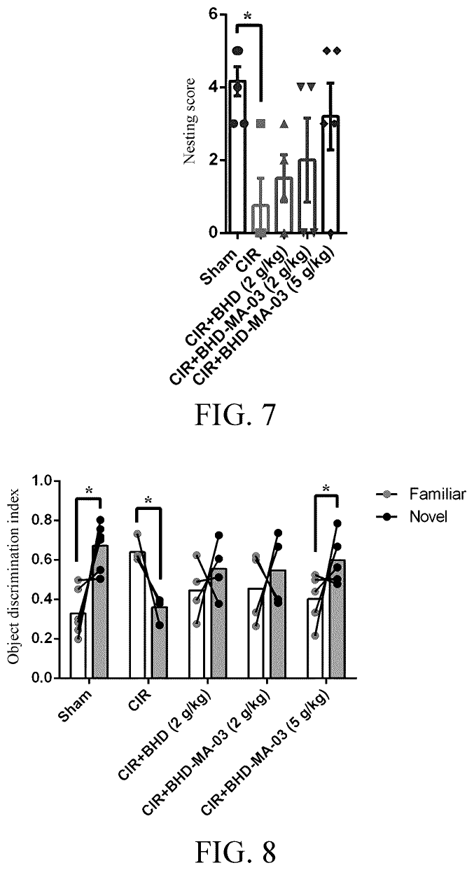

The mice in each group underwent a daily living ability test 7 days after being induced with acute stroke (the test was performed on the surviving mice in each group). The test method was the same as that of the aforementioned daily living ability test. The test results are shown in FIG. 7. The average score of the mice in the sham group could reach about 4.2 points, the average score of the mice in the BHD-MA-03 (5 g/kg) (NRICM301) group could reach 3 points, the average score of the mice in the BHD-MA-03 (2 g/kg) (NRICM301) group could reach 2 points, the average score of the mice in the CIR+BHD (2 g/kg) (ancient prescription) group could reach 1.5 points, and the average score of the mice in the CIR group was about 1 point. According to the above test results, it could be known that NRICM301 had an effect of gradually and continuously improving the daily living ability of mice after stroke.

The mice in each group underwent a learning and memory ability test 7 days after being induced with acute stroke (the test was performed on the surviving mice in each group). The test method was described as follows. First, two similar objects (referred to as “Familiar”) were given to the mice in each group for identification, and the time spent by the mice in each group in identifying these two similar objects was calculated. The next day, one of the similar objects was replaced with a novel object (referred to as “Novel”), and the time spent by the mice in each group in identifying the similar object and the novel object separately was calculated. Finally, the time spent by the mice in each group on the second day in identifying the similar object and the novel object was converted into a discrimination index. The test results are shown in FIG. 8. The mice in the sham group (normal mice) spent more time identifying the novel object on the second day and less time on the similar object that had been identified the previous day. This indicated that normal mice had the learning and memory ability to learn and memorize old objects. The mice in the BHD-MA-03 (5 g/kg) (NRICM301) group, the BHD-MA-03 (2 g/kg) (NRICM301) group and the CIR+BHD (2 g/kg) (ancient prescription) group showed a similar trend to that of the mice in the sham group. However, the data of the mice in the CIR group were the opposite of those of the aforementioned groups. This meant that the mice after stroke lost their learning and memory ability, and both NRICM301 and its ancient prescription were helpful for the recovery of the learning and memory ability of mice after stroke.

### Tissue Section Experiment on the Therapeutic Effect of NRICM301 on Acute Stroke

First, the aforementioned test process of the behavioral observation test on the therapeutic effect of NRICM301 on acute stroke was reproduced, and on the 7th day after the mice in each group started the test, the mice in each group were sacrificed, and their brains were sectioned to observe the therapeutic effect of NRICM301 and the ancient prescription on the mice after acute stroke. The specific experimental process was described as follows.

First, the mice in the sham group (normal mice), the CIR group (mice after stroke induction), the BHD-MA-03 (5 g/kg) (NRICM301) group and the CIR+BHD (2 g/kg) (ancient prescription) group were anesthetized with a mixture of isoflurane at a concentration of 1.5-2% wt and oxygen, and then systemic perfusion was performed to sacrifice the mice in each group in this way. The method of systemic perfusion was to first conduct systemic washing of the mice in each group with a phosphate buffer solution, and then complete fixation perfusion of the mice in each group with a phosphate buffer solution containing 4% paraformaldehyde to sacrifice them.

After the mice in each group were sacrificed, the brains of the mice in each group were removed. After the brains were removed, the brain samples of the mice in each group were placed in a phosphate buffer solution containing 4% paraformaldehyde for fixation overnight. Then, the brain samples of the mice in each group were separately placed in phosphate buffer solutions containing 10%, 20% and 30% sucrose at 4° C. The brain samples of the mice in each group at each concentration were dehydrated for one day. After the brain samples of the mice in the aforementioned groups were dehydrated, they were embedded in Tissue-Tek OCT frozen embedding medium (purchased from Sakura Finetek, Torrance, California, USA), and the brain samples of the mice in the aforementioned groups were frozen in liquid nitrogen. Subsequently, the brain samples of the mice in the aforementioned groups were made into coronal brain sections with a thickness of 30 μm (in the range of −1.5 to −1.9 mm from the bregma site) by using a freezing microtome (Microm HM560, purchased from Walldorf, Heidelberg, Germany).

After the brain sections of the mice in each group were prepared, the sections were placed in an immunohistochemical (IHC) buffer solution containing Triton, Tween-20 and NaN₃ and 2% serum, and left overnight at 4° C. to block the section tissues. The next day, the brain sections of the mice in each group were washed once with the IHC buffer solution for 5 minutes each time.

Then, immunohistochemical staining of the brain sections of the mice in each group was performed. The brain sections of the mice in each group were added with appropriate primary antibodies and cultured at 4° C. for 2-3 days. The primary antibodies used included: CD206 (1:200; ir240-864, purchased from iREAL, Taipei, Taiwan); CD68 (1:50, purchased from BD, San Diego, California, USA); Doublecortin (DCX) (1:1000, AB2253, purchased from Merck-Millipore, Taipei, Taiwan); and IBA1 (1:100, ab5076, purchased from Abcam, Cambridge, UK). After the brain sections of the mice in each group were cultured with the primary antibodies, they were washed 6 times, and then added with appropriate secondary antibodies (Alexa Fluor 488, 555 or 647, 1:200; purchased from Cell Signaling Technology, Danvers, Massachusetts, USA) and cultured at 4° C. for 2-3 days to complete the immunohistochemical staining.

After the immunohistochemical staining of the brain sections of the mice in each group was completed, the brain sections were washed 6 times again. The slides containing the brain sections of the mice in each group were added with mounting medium and covered with cover slips. The edges of the cover slips were fixed with nail polish to complete the slide mounting.

After the mounting of the brain sections of the mice in each group was completed, the sections were observed using a confocal microscope (Zeiss LSM780, purchased from Carl Zeiss AG, Jena, Germany), and data analysis was performed using imaging software (Zen2011, purchased from Carl Zeiss AG, Germany).

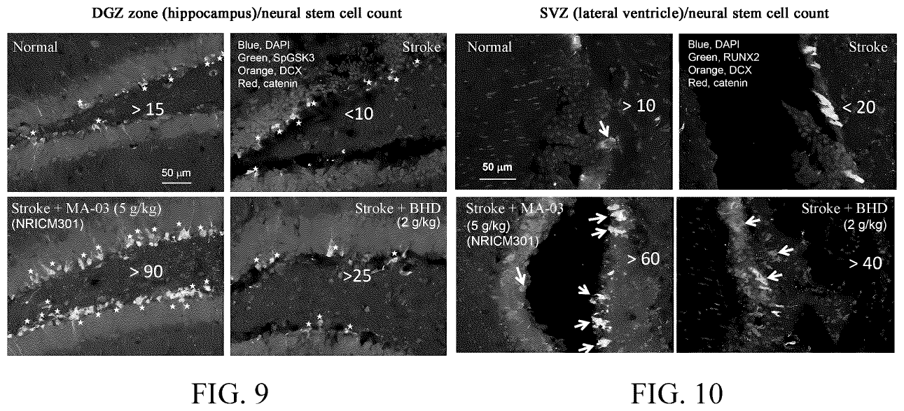

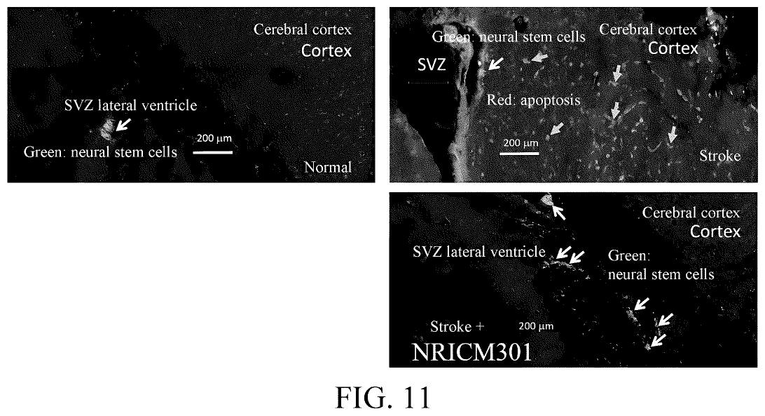

The results of this test 4 are shown in FIGS. 9-11. Compared with the mice in the CIR group, the regeneration of nerve cells could be observed in the brain sections of the mice in both the BHD-MA-03 (5 g/kg) (NRICM301) group and the CIR+BHD (2 g/kg) (ancient prescription) group.

According to the above test results, the food and medicine compound medicament provided in this example could improve the sequelae of acute central nervous system injury, especially the walking ability, daily living ability and learning and memory ability of mice. Therefore, the above food and medicine component composition or the food and medicine compound medicament prepared therefrom could be used to prepare drugs for treating acute central nervous system injury or the sequelae of acute central nervous system injury.

The present invention has been disclosed above with the preferred example. However, those skilled in the art should understand that the example is merely used to describe the present invention and should not be construed as limiting the scope of the present invention. It should be noted that all changes and substitutions equivalent to the example should be encompassed within the scope of the present invention. Therefore, the scope of protection for the present invention shall be defined by the claims.

What is claimed is:

1. A food and medicine component composition, comprising:
0.5 to 1.5 parts by weight of Red Peony Root;
0.5 to 1.5 parts by weight of Chuanxiong Rhizome;
10 to 15 parts by weight of Hedysarum Root; and
50 to 55 parts by weight of whole raw fish.

2. The food and medicine component composition according to claim 1, wherein the whole raw fish is selected from the group consisting of Monopterus and Anguilla.

3. The food and medicine component composition of claim 1, wherein a species of the whole raw fish is Monopterus albus.

4. The food and medicine component composition according to claim 1, wherein parts by weight of the whole raw fish are 52 to 54 parts by weight.

5. A method for preparing a food and medicine compound medicament, comprising:
(a) providing 0.5 to 1.5 parts by weight of Red Peony Root, 0.5 to 1.5 parts by weight of Chuanxiong Rhizome, 10 to 15 parts by weight of Hedysarum Root, 50 to 55 parts by weight of whole raw fish and 200 to 400 parts by weight of water;
(b) soaking the Red Peony Root, the Chuanxiong Rhizome, the Hedysarum Root and the whole raw fish in the water, and heating the water to boiling to perform decocting treatment on the Red Peony Root, the Chuanxiong Rhizome, the Hedysarum Root and the whole raw fish; and
(c) boiling the water provided in step (a) to approximately 75% by volume of an initial volume of the water to obtain the food and medicine compound medicament.

6. The preparation method according to claim 5, wherein the whole raw fish is selected from the group consisting of Monopterus and Anguilla.

7. The preparation method according to claim 5, wherein a species of the whole raw fish is Monopterus albus.

8. A food and medicine compound medicament prepared by the preparation method according to claim 5.

9. A method for treating acute central nervous system injury or a sequela of acute central nervous system injury, comprising administrating a drug prepared from the food and medicine component composition which includes the food and medicine component composition according to claim 1 to a subject in need thereof.

10. A method for treating acute central nervous system injury or a sequela of acute central nervous system injury, comprising administrating a food and medicine compound medicament which includes the food and medicine compound medicament according to claim 8 to a subject in need thereof.
